(19) United States
(12) Patent Application Publication
(10) Pub. No.: US 2010/0305500 A1
(43) Pub. Date: Dec. 2, 2010

(54) HYALURONIDASE AS AN ADJUVANT FOR INCREASING THE INJECTION VOLUME AND DISPERSION OF LARGE DIAMETER SYNTHETIC MEMBRANE VESICLES CONTAINING A THERAPEUTIC AGENT

(75) Inventors: William LAMBERT, San Diego, CA (US); Jason REXROAD, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: PACIRA PHARMACEUTICALS, INC., San Diego, CA (US)

(21) Appl. No.: 12/790,587

(22) Filed: May 28, 2010

Related U.S. Application Data
(60) Provisional application No. 61/182,367, filed on May 29, 2009.

(51) Int. Cl. A61K 9/127; A61K 31/711; A61K 38/19; A61K 38/18; A61K 38/24; A61P 5/06; A61P 9/06; A61P 11/06; A61P 31/10; A61P 3/10; A61P 9/12; A61P 35/00; A61P 31/12; A61P 25/24; A61P 25/18; A61P 33/02; A61P 33/10; A61M 5/31

(52) U.S. Cl. 604/82; 424/450; 514/44 R; 424/85.1; 514/8.4; 514/9.1; 514/8.2; 514/10.1; 604/191

(57) ABSTRACT

Embodiments of the invention relate to hyaluronidase as an adjuvant to increase the injection volume and dispersion of large diameter synthetic membrane vesicles containing one or more therapeutic agents. In particular, embodiments of the invention relate to compositions comprising hyaluronidase and large diameter synthetic membrane vesicles containing a therapeutic agent, and methods of administration of the same. Methods of making large diameter synthetic membrane vesicles containing an active pharmaceutical ingredient and their use in combination with hyaluronidase as medicaments are provided.

This application claims priority under 35 U.S.C. §119(e)(1) to U.S. Patent Application No. 61/182,367, filed May 29, 2009.

### BACKGROUND OF THE INVENTION

### Field of the Invention

Embodiments of the invention relate to hyaluronidase as an adjuvant to increase the injection volume and dispersion of large diameter synthetic membrane vesicles containing one or more therapeutic agents. In particular, embodiments of the invention relate to compositions comprising hyaluronidase and large diameter synthetic membrane vesicles containing a therapeutic agent, and methods of administration of the same. Methods of making large diameter synthetic membrane vesicles containing an active pharmaceutical ingredient and their use in combination with hyaluronidase as medicaments are provided.

Large Diameter Synthetic Membrane Vesicles

Large diameter synthetic membrane vesicles include multivesicular liposomes, first reported by Kim, et al. (Biochim, Biophys. Acta, 728:339-348, 1983), as well as other lipid-based delivery systems such as unilamellar (Huang, Biochemistry, 8:334-352, 1969; Kim, et al., Biochim. Biophys. Acta, 646:1-10, 1981) and multilamellar (Bangham, et al., J Mol. Bio., 13:238-252, 1965) liposomes. The main structural difference between multivesicular liposomes and unilamellar liposomes (also known as unilamellar vesicles), is that multivesicular liposomes contain multiple aqueous chambers per particle. The main structural difference between multivesicular liposomes and multilamellar liposomes (also known as multilamellar vesicles), is that in multivesicular liposomes the multiple aqueous chambers in multivesicular liposomes are non-concentric. The structural differences between unilamellar, multilamellar, and multivesicular liposomes are illustrated in U.S. Pat. No. 5,766,627 and U.S. Pat. No. 6,132,766, incorporated herein by reference in their entireties. In some embodiments, Large diameter synthetic membrane vesicles can be prepared as disclosed in U.S. Pat. No. 6,045,824, Intl. Pub. No. WO 99/13865, and Intl. Pub. No. WO 99/25319 incorporated herein by reference in their entireties.

The structural and functional characteristics of multivesicular liposomes are not directly predictable from current knowledge of unilamellar vesicles and multilamellar vesicles. Multivesicular liposomes are bounded by an external bilayer membrane shell, but have a very distinctive internal morphology, which may arise as a result of the special method employed in the manufacture. Topologically, multivesicular liposomes are defined as having multiple non-concentric chambers within each particle, resembling a “foam-like” matrix; whereas multilamellar vesicles contain multiple concentric chambers within each liposome particle, resembling the “layers of an onion.”

The presence of internal membranes distributed as a network throughout multivesicular liposomes may serve to confer increased mechanical strength to the vesicle, while still maintaining a high volume:lipid ratio compared with multilamellar vesicles. The multivesicular nature of multivesicular liposomes also indicates that, unlike for unilamellar vesicles, a single breach in the external membrane of a synthetic membrane vesicleswill not result in total release of the internal aqueous contents.

Hyaluronidase

Hyaluronidase, a mucolytic enzyme used as a “spreading factor,” hydrolyzes hyaluronan, a megadalton glycosaminoglycan found in connective tissue (Chain et al.: Identity of hyaluronidase as spreading factor, Br J Exp Pathol 1940; 21. and Myers K: Biological significance of hyaluronic acid and hyaluronidase, Physiol Rev 1947; 27.). This hydrolysis reduces the viscosity of the gellike interstitial matrix, effecting an increase in the diffusion and absorption of subcutaneously administered fluids (Hechter O: The clinical use of hyaluronidase in hypodermoclysis, J Pediatr 1947; 30:645-656. and Lipschitz et al.: Subcutaneous fluid administration in elderly subjects: Validation of an underused technique, J Am Geriatr Soc 1991; 39:6-9.) and a decrease in infusion site swelling (Webb W: Effect of hyaluronidase on rate of absorption of subcutaneous fluids, Arch Surg 1952: 770-773. and Constans et al.: Hypodermoclysis in dehydrated elderly patients: Local effects with and without hyaluronidase, J Palliat Care 1991; 7:10-12.). Methods of preparation and use of a human hyaluronidase is illustrated in U.S. Pat. No. 7,105,330 and U.S Pub. No. 2004/0268425, incorporated herein by reference in their entireties.

### SUMMARY OF THE INVENTION

The present embodiments provide a pharmaceutical composition for enhancing the administration of a therapeutic agent in large diameter synthetic membrane vesicles, comprising hyaluronidase; and large diameter synthetic membrane vesicles, the large diameter synthetic membrane vesicles having encapsulated therein a therapeutic agent. In some embodiments, the therapeutic agent is a therapeutic peptide or a proteinaceous material. In some embodiments, the hyaluronidase is also encapsulated in large diameter synthetic membrane vesicles, optionally the same vesicles as that contain the therapeutic agent. In some embodiments, the large diameter synthetic membrane vesicles have an average diameter of at least 500 nm. Further embodiments provide a pharmaceutical composition where the large diameter synthetic membrane vesicles have an average diameter between 1 μm and 50 μm. Another embodiment provides the pharmaceutical composition of any of the disclosed embodiments where the hyaluronidase is rHuPH20.

The present embodiments provide a method of administering a therapeutic agent to a subject in need thereof, comprising administering the pharmaceutical compositionsdisclosed herein to the subject. In some embodiments, the composition is administered by injection. In other embodiments, the administration is selected from the group consisting of subcutaneous injection or infusion, intramuscular injection or infusion and intradermal injection or infusion.

The present embodiments provide a method of enhancing the administration of a therapeutic agent in large diameter synthetic membrane vesicles to a subject in need thereof, comprising administering hyaluronidase to a subject; and, administering large diameter synthetic membrane vesicles having encapsulated therein a therapeutic agent to the subject. In a preferred embodiment, the ease of administration of the large diameter synthetic membrane vesicles is improved, or the volume of the large diameter synthetic membrane vesicles that can be administered is increased, in comparison to administration of the large diameter synthetic membrane vesicles in the absence of the hyaluronidase. In some embodiments, the hyaluronidase and the large diameter synthetic membrane vesicles are administered concurrently. In some embodiments, the hyaluronidase and the large diameter synthetic membrane vesicles are administered as a mixture.

In some embodiments, the site of administration of the hyaluronidase and the site of administration of the large diameter synthetic membrane vesicles are the same. In other embodiments, the site of administration of the hyaluronidase and the site of administration of the large diameter synthetic membrane vesicles are less than 5 cm from each other.

Some embodiments provide a method further comprising administering additional large diameter synthetic membrane vesicle encapsulating a therapeutic agent to the subject after a period of time following the initial administration of the hyaluronidase and the large diameter synthetic membrane vesicles, for example following their concurrent administration. In some embodiments, administration of the large diameter synthetic membrane vesicles follows administration of the hyaluronidase, the concurrent administration of hyaluronidase and large diameter synthetic membrane vesicles, or a previous administration of large diameter synthetic membrane vesicles, after a period of time. In some embodiments, the period of time is at least about 1 minute.

In some embodiments, the administration of the hyaluronidase and the large diameter synthetic membrane vesicles is selected from the group consisting of subcutaneous, intramuscular and intradermal. In some embodiments the hyaluronidase is administered by bolus injection or infusion and the large diameter synthetic membrane vesicles are administered by bolus injection or infusion. In some embodiments, the hyaluronidase is encapsulated in large diameter synthetic membrane vesicles, either alone or in the same vesicles as the therapeutic agent.

Preferred embodiments provide a method of where the large diameter synthetic membrane vesicles have an average diameter of at least 500 nm. In some embodiments, the large diameter synthetic membrane vesicles have an average diameter between 1 μm and 50 μm. In some embodiments, the hyaluronidase is rHuPH20.

Another embodiment of the present invention provides a kit for enhancing the administration of a therapeutic agent in large diameter synthetic membrane vesicles comprising a first sterile container comprising large diameter synthetic membrane vesicles, the large diameter synthetic membrane vesicles having encapsulated therein a therapeutic agent, and, a second sterile container comprising hyaluronidase. In some embodiments, the first and second containers are chambers of a two chamber syringe. In other embodiments, the kit further comprises a third container for mixing the hyaluronidase and the large diameter synthetic membrane vesicles.

Some embodiments provide a kit of any of the disclosed embodiments where the hyaluronidase is rHuPH20. Other embodiments provide a kit of any of the disclosed embodiments, where the large diameter synthetic membrane vesicles have an average diameter of at least 500 nm. Other embodiments provide a kit where the large diameter synthetic membrane vesicles have an average diameter between 1 μm and 50 μm.

The present embodiments provide a method of providing a therapeutic agent in large diameter synthetic membrane vesicles to an individual comprising, providing the individual large diameter synthetic membrane vesicles, the large diameter synthetic membrane vesicles having encapsulated therein a therapeutic agent, and providing the individual instructions to administer hyaluronidase prior to or concurrent with the large diameter synthetic membrane vesicles. In some embodiments, the large diameter synthetic membrane vesicles have an average diameter of at least 500 nm. In other embodiments, the large diameter synthetic membrane vesicles have an average diameter between 1 μm and 50 μm. Some embodiments provide a method of any of the preceding embodiments where the hyaluronidase is rHuPH20.

### DETAILED DESCRIPTION OF THE PREFERRED EMBODIMENT

Multivesicular liposomes formulations consist of microscopic, spherical particles composed of numerous nonconcentric aqueous chambers encapsulating the drug to be delivered. The individual chambers are separated by lipid bilayer membranes composed of synthetic duplicates of naturally occurring lipids, resulting in a delivery vehicle that is both biocompatible and biodegradable. The multivesicular liposomes formulations provide either local site or systemic sustained delivery, and can be administered by a number of routes including under the skin, into muscle tissue, into spinal fluid, joints, and the abdominal cavity. Typically, a multivesicular liposome particle consists of approximately 10% lipid and 90% percent aqueous drug solution and can be approximately 20 μm in diameter. Preparation of multivesicular liposomes are illustrated in U.S. Pat. No. 5,766,627 and U.S. Pat. No. 6,132,766, incorporated herein by reference in their entireties.

Hyaluronidase, such as the recombinant formulation of hyaluronidase marketed under HYLENEX™ has received FDA market approval as of December 2005 as an adjuvant to increase the absorption and dispersion of injected drugs. The enzyme acts by cleaving hyaluronan present in the interstitial collagenous matrix and results in less extracellular matrix resistance. Following cleavage, the effects of hyaluronidase are completely reversible within 24 h of injection. Hyaluronidase has been evaluated for efficacy of dispersion with particles from 20 nm to 1 μm. However, the dispersion of 500 nm and 1 μm diameter particles in the presence of hyaluronidase was not significantly different from carrier controls up to 4 h post-injection, indicating that hyaluronidase treatment did not improve the diffusion of agents larger than 200 nm (Bookbinder et al.: A recombinant human enzyme for enhanced interstitial transport of therapeutics, Journal of Controlled Release 114 (2006) 230-241). Methods of preparation and use of a human hyaluronidase are illustrated in U.S. Pat. No. 7,105,330 and U.S Pub. No. 2004/0268425, incorporated herein by reference in their entireties.

Subcutaneous injections are generally limited to 2.5 mL, and more often less than 1 mL. This limit is due to the wheal that forms from the injected volume and the increasing pressure required to inject larger volumes due to tissue resistance. This volume restriction therefore limits the amount of therapeutic agent which can be delivered. Additionally, large diameter synthetic membrane vesicles, can further limit the volume of injection since the large diameter synthetic membrane vesicle particles can become trapped in the interstitial spaces, effectively clogging delivery channels into the tissue and temporarily preventing further injection at that site. As a result, multiple injections of formulations including large diameter synthetic membrane vesicles or new formulations may be necessary to provide a therapeutically effective amount of the therapeutic agent.

Surprisingly, it has been found that administration of hyaluronidase, such as recombinant human hyaluronidase, allows for larger volumes of large diameter synthetic membrane vesicles, such as multivesicular liposomes, having a particle size greater than about 500 nm diameter, to be administered, and the ease of administration is improved. Prior to the present disclosure, it was believed that particles with diameters of larger than 200 nm would not manifest increased dispersion in the presence of hyaluronidase. As discussed previously, dispersion of 500 nm and 1 μm diameter particles in the presence of recombinant human hyaluronidase was not significantly different from carrier controls up to 4 h post-injection, indicating that recombinant human hyaluronidase treatment did not improve the diffusion of agents larger than 200 nm. Thus, the present findings that a decreased force of injection was required with multivesicular liposomes formulations co-administered with recombinant human hyaluronidase were unexpected since the mean diameter of the multivesicular liposomes is approximately 100-fold larger than the previously reported upper limit of particle size assisted by hyaluronidase treatment. Additionally, volumes greater than 2.5 mL of the large diameter synthetic liposomes formulations were injected subcutaneously, obviating the need to use multiple injections or overcome formulation challenges related to efforts to deliver more therapeutic agent. In addition to increasing in amount of therapeutic agent permitted to be injected, the bioavailability and pharmacokinetics of the therapeutic agent can be improved by the effect of the hyaluronidase, requiring less drug to achieve the same therapeutic effect. Accordingly, the amount of therapeutic agent required can be reduced, in some embodiments.

Although the embodiments and examples disclosed herein refer to recombinant human hyaluronidase, in any of the embodiments herein, it is contemplated that the hyaluronidase can be any hyaluronidase that is appropriate for administration to a subject. For example, the hyaluronidase can be derived from animal, human and/or bacterial sources including naturally occurring and recombinant forms. Examples of animal-derived hyaluronidase include, but are not limited to, HYDASE™, VITRASE®, AMPHADASE® and WYDASE. Examples of a human-derived hyaluronidase include, but are not limited to, HYLENEX™. Also envisioned for use in any of the embodiments disclosed herein are active enzymatic fragments of a hyaluronidase, or mixtures of one or more hyaluronidase and/or active fragments thereof.

Some embodiments relate to a pharmaceutical formulation including recombinant human hyaluronidase encapsulated in synthetic membrane vesicles. In some embodiments, the pharmaceutical formulation optionally includes a pharmaceutically acceptable carrier.

Some embodiments provide a pharmaceutical composition, comprising synthetic membrane vesicles, having encapsulated therein a therapeutic agent; and a recombinant human hyaluronidase, optionally encapsulated in the same or different large diameter synthetic membrane vesicles. In some embodiments the recombinant human hyaluronidase is rHuPH20.

Some embodiments provide a pharmaceutical composition, comprising synthetic membrane vesicles, having encapsulated therein a recombinant human hyaluronidase. In some embodiments, the synthetic membrane vesicles, having encapsulated therein a recombinant human hyaluronidase, additionally have one or more therapeutic agents encapsulated therein. In some embodiments, the one or more therapeutic agents can be selected from the group consisting of peginterferon alfa-2a, bevacizumab and trastuzumab. In some embodiments the recombinant human hyaluronidase is rHuPH20.

Some embodiments relate to administration of recombinant human hyaluronidase and large diameter synthetic membrane vesicles together to a subject.

Some embodiments relate to administration of recombinant human hyaluronidase to a subject followed by administration of large diameter synthetic membrane vesicles having encapsulated therein a therapeutic agent to the subject.

Some embodiments provide a method of administering a therapeutic agent to a subject, comprising administering recombinant human hyaluronidase to a subject, wherein the hyaluronidase is optionally encapsulated in large diameter synthetic membrane vesicles, and administering large diameter synthetic membrane vesicles having encapsulated therein a therapeutic agent to the subject. In some embodiments the hyaluronidase and the large diameter synthetic membrane vesicles encapsulating a therapeutic agent are administered concurrently. In some embodiments, the recombinant human hyaluronidase is administered to a subject and then large diameter synthetic membrane vesicles having encapsulated therein a therapeutic agent are administered to the subject. In some embodiments, a first, second, third, fourth, fifth or subsequent amount of large diameter synthetic membrane vesicles having encapsulated therein a therapeutic agent are administered to a subject from about 1 second to about 60 seconds, about 1 minute to about 5 minutes, about 5 minutes to about 10 minutes, about 10 minutes to about 30 minutes, about 30 minutes to about 60 minutes, about 1 hour to about 3 hours, about 2 to about 6 hours, about 6 to about 12 hours, about 10 to about 18 hours, or about 6 to 24 hours, after administration of the recombinant human hyaluronidase. In some embodiments, the first, second, third, fourth, fifth or subsequent amount of large diameter synthetic membrane vesicles having encapsulated therein a therapeutic agent are administered to a subject not more than about 1, 2, 3, 4, 5, 7, 10, 12, 15, 18, 20, 22 or 24 hours after administration of the hyaluronidase. In a preferred embodiment, the administration of hyaluronidase is by subcutaneous injection or infusion. In a preferred embodiment, the administration of the large diameter synthetic membrane vesicles is by subcutaneous injection or infusion.

Some embodiments provide a method of administering a therapeutic agent to a subject, comprising injecting a solution of hyaluronidase into the subject; and injecting large diameter synthetic membrane vesicles into the subject, having encapsulated therein a therapeutic agent. In some embodiments, the hyaluronidase is injected subcutaneously into the subject and the large diameter synthetic membrane vesicles having encapsulated therein a therapeutic agent are injected subcutaneously into the subject. In some embodiments, the injection sites are the same, and in some embodiments the injection sites are less than 1 cm from each other. In some embodiments, the injection sites are less than 5 cm from each other. In some embodiments, the injection sites are less than 10 cm from each other.

Some embodiments provide a method of administering in a single injection a therapeutic agent to a subject, comprising injecting at least about 1 mL, 2 mL, 3 mL, 4 mL, 5 mL, 10 mL, 15 mL, or 20 mL of large diameter synthetic membrane vesicles, having encapsulated therein the therapeutic agent. In a typical embodiment, the volume of large diameter synthetic membrane vesicles, having encapsulated therein the therapeutic agent is at least 3 mL, 4 mL, 5 mL, 10 mL, 15 mL, or 20 mL. Some embodiments provide a method of administering by infusion a therapeutic agent to a subject, comprising infusing at least about 10 mL, 15 mL, 20 mL, 50 mL, 100 mL, or 1000 mL of large diameter synthetic membrane vesicles, having encapsulated therein the therapeutic agent. In some embodiments the flow rate of the infusion is about 10, 20, 50, 100, 200, 300, 400, 500 or more mL/hour. In some embodiments, hyaluronidase is administered subcutaneously into the subject prior to administering large diameter synthetic membrane vesicles, having encapsulated therein a therapeutic agent. In some embodiments, hyaluronidase and large diameter synthetic membrane vesicles, having encapsulated therein the therapeutic agent are administered simultaneously.

Some embodiments provide a method of preparing a medicament or pharmaceutical composition for administration to a subject comprising mixing recombinant human hyaluronidase and synthetic membrane vesicles, the large diameter synthetic membrane vesicles having encapsulated therein a therapeutic agent. Some embodiments provide a method of administering a therapeutic agent to a subject, comprising mixing recombinant human hyaluronidase and synthetic membrane vesicles, the large diameter synthetic membrane vesicles having encapsulated therein a therapeutic agent; and injecting the mixture of the recombinant human hyaluronidase and large diameter synthetic membrane vesicles into the subject. In some embodiments, the recombinant human hyaluronidase is rHuPH20. In some embodiments, the mixture containing the recombinant human hyaluronidase, large diameter synthetic membrane vesicles and therapeutic agent is injected subcutaneously into a subject. In some embodiments, the mixture containing the recombinant human hyaluronidase, large diameter synthetic membrane vesicles and therapeutic agent is injected subcutaneously into a subject from about 1 second to about 60 seconds, 1 minute to about 5 minutes, about 5 minutes to about 10 minutes, about 10 minutes to about 30 minutes, about 30 minutes to about 60 minutes, or about 1 hour to about 3 hours, after mixing.

Some embodiments provide a syringe comprising a pharmaceutical composition comprising a recombinant human hyaluronidase and large diameter synthetic membrane vesicles, having encapsulated therein a therapeutic agent.

Some embodiments provide a syringe comprising, a recombinant human hyaluronidase, large diameter synthetic membrane vesicles and a therapeutic agent. In some embodiments, the syringe has a single chamber containing the recombinant human hyaluronidase, large diameter synthetic membrane vesicles and the therapeutic agent. In some embodiments the syringe has two chambers wherein the large diameter synthetic membrane vesicles and therapeutic agent can be in one chamber and the recombinant human hyaluronidase can be in the other chamber.

Some embodiments provide a sterile, optionally single use container, comprising a pharmaceutical composition comprising; large diameter synthetic membrane vesicles having encapsulated therein a therapeutic agent; and a recombinant human hyaluronidase.

Some embodiments provide a kit comprising: a sterile, optionally single use container, comprising a pharmaceutical composition, comprising a recombinant human hyaluronidase and large diameter synthetic membrane vesicles having encapsulated therein a therapeutic agent; and a syringe adapted to receive solution from the container. Some embodiments provide a kit comprising: a sterile, optionally single use, container comprising synthetic membrane vesicles, having encapsulated therein a therapeutic agent, and recombinant human hyaluronidase. Some embodiments provide a kit comprising: a sterile, optionally single use, container comprising synthetic membrane vesicles, having encapsulated therein a therapeutic agent, and a sterile, optionally single use, container comprising recombinant human hyaluronidase. In some embodiments, the containers are chambers of a multi-chamber vessel.

Some embodiments provide a method of providing a therapeutic agent in large diameter synthetic membrane vesicles to an individual comprising providing said individual large diameter synthetic membrane vesicles, said large diameter synthetic membrane vesicles having encapsulated therein a therapeutic agent, and providing said individual instructions to administer hyaluronidase prior to or concurrent with said large diameter synthetic membrane vesicles. Some embodiments provide a method wherein a subject is instructed to administer large diameter synthetic membrane vesicles, said large diameter synthetic membrane vesicles having encapsulated therein a therapeutic agent. Some embodiments provide a method wherein a subject is instructed to administer large diameter synthetic membrane vesicles, said large diameter synthetic membrane vesicles having encapsulated therein a therapeutic agent, and hyaluronidase. In some embodiments, the instruction to administer is provided in writing, optionally on a label or package insert associated with a container comprising said large diameter synthetic membrane vesicles having encapsulated therein a therapeutic agent. In some embodiments, the label is a government approved and, or, required label, for example a label required by the Food and Drug Administration of the U.S.

Some embodiments provide for the manufacture of medicaments according to or for use with any of the compositions, kits and methods described herein.

As used herein, the term “subject” includes animals and humans. In a preferred embodiment, the subject is a human.

### Methods of Administration

In any of the embodiments, administration can be by bolus injection, e.g., subcutaneous bolus injection, intramuscular bolus injection, intradermal bolus injection and the like. In any of the embodiments, administration can be by infusion, e.g., subcutaneous infusion, intramuscular infusion, intradermal infusion, and the like. In some embodiments, administration can be by direct tissue injections, including injections into tumors and masses.

Administration, preferably subcutaneous administration, of a pharmaceutical composition of the embodiments is accomplished using standard methods and devices, e.g., pens, injector systems, needle and syringe, a subcutaneous injection port delivery system, and the like. See, e.g., U.S. Pat. Nos. 3,547,119; 4,755,173; 4,531,937; 4,311,137; and 6,017,328, each of which is herein incorporated by reference in their entirety. A combination of a subcutaneous injection port and a device for administration of a pharmaceutical composition of the embodiments to a patient through the port is referred to herein as “a subcutaneous injection port delivery system.” In any of the embodiments, subcutaneous administration can be achieved by bolus delivery by needle and syringe.

Solutions or suspensions used for parenteral, intradermal, subcutaneous, intramuscular or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; cheating agents, such as ethylenediaminetetraacetic acid (EDTA); buffers, such as acetates, citrates and phosphates; and agents for the adjustment of tonicity, including, but not limited to sodium chloride, calcium chloride, magnesium chloride, dextrose, glycerol or boric acid. Parenteral preparations can be enclosed in ampoules, disposable syringes or single or multiple dose vials made of glass, plastic or other suitable material.

Parenteral administration of hyaluronidase, generally characterized by injection, either subcutaneously, intradermally, or intramuscularly is also contemplated herein. In some embodiments, parenteral administration of hyaluronidase includes direct tissue injections, including injections into tumors and masses. Injectables can be prepared in conventional forms, either as liquid solutions or suspensions; solid forms suitable for solution or suspension in liquid prior to injection, or as emulsions. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as, for example, sodium acetate, sorbitan monolaurate, triethanolamine oleate and cyclodextrins.

Parenteral administration of the compositions includes subcutaneous intramuscular, and intradermal administrations. In some embodiments, parenteral administration of the compositions includes direct tissue injections, including injections into tumors and masses. Preparations for parenteral administration include sterile solutions ready for injection, sterile dry soluble products, such as lyophilized powders, ready to be combined with a solvent or sterile solution just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thiomersal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the therapeutic agent and/or hyaluronidase can be adjusted so that administration, e.g., an injection or infusion, provides an effective amount to produce the desired pharmacological effect. The exact dose depends on the age, weight and condition of the patient or animal as is known in the art.

The unit-dose parenteral preparations can be packaged in container, for example, an ampoule, a vial or a syringe with a needle. All preparations for parenteral administration must be sterile, as is known and practiced in the art.

Illustratively, subcutaneous infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The hyaluronidase can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compounds provided herein can be formulated for parenteral administration by injection, e.g., by bolus injection or continuous infusion. Formulations for injection can be presented in unit dosage form, e.g., in ampoules or in multi-dose containers, optionally with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use. For example, provided herein are parenteral formulations containing an effective amount of hyaluronidase, such as 0.5 to 500,000 Units, in a stabilized solution or a lyophilized from.

Some embodiments also include pharmaceutical formulations for parenteral administration, e.g., by bolus injection or continuous infusion, include aqueous suspensions of the synthetic membrane vesicles, having encapsulated therein a therapeutic agent. Additionally, suspensions of the large diameter synthetic membrane vesicles may be prepared as appropriate. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. In some embodiments the formulation can be administered by subcutaneous injection.

The effective daily dosage of the therapeutic agent may be varied over a wide range; e.g., from about 0.1 μg to about 10,000 mg per adult human per day.

Therapeutically effective dosages of a therapeutic agent can range from 0.1 μg to about 0.5 μg per dose, from about 0.5 μg to about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, from about 35 mg to about 40 mg per dose, from about 40 mg to about 50 mg per dose, from about 50 mg to about 75 mg per dose, from about 75 mg to about 100 mg per dose, from about 100 mg to about 200 mg per dose, from about 200 mg to about 400 mg per dose, from about 400 mg to about 600 mg per dose, from about 600 mg to about 800 mg per dose, or from about 800 mg to about 5000 mg per dose. An effective amount of the instant compounds is ordinarily supplied at a dosage level of from about 0.01 mg/kg to about 150 mg/kg of body weight per day. Typically, the range is from about 0.1 to about 80 mg/kg of body weight per day, and especially from about 0.2 mg/kg to about 40 mg/kg of body weight per day. The compounds may be administered on a regimen of about 1 to about 10 times per day, or continuous infusion for a period of from about 1, 5, 10, 15, 30 or 60 minutes, or 1, 2, 3, 5, 7, 10, 12, 15, 18, 20 or 24 hours or a range defined by any two of the preceding values.

Effective dosages of recombinant human hyaluronidase can range from 0.1 United States Pharmacopeia (USP) units to about 0.5 USP units per dose, from about 0.5 USP units to about 1.0 USP units per dose, from about 1.0 USP units per dose to about 5.0 USP units per dose, from about 5.0 USP units to about 10 USP units per dose, from about 10 USP units to about 20 USP units per dose, from about 20 USP units per dose to about 30 USP units per dose, from about 30 USP units per dose to about 40 USP units per dose, from about 40 USP units per dose to about 50 USP units per dose, from about 50 USP units per dose to about 60 USP units per dose, from about 60 USP units per dose to about 70 USP units per dose, from about 70 USP units to about 80 USP units per dose, from about 80 USP units per dose to about 100 USP units per dose, from about 100 USP units to about 150 USP units per dose, from about 150 USP units to about 200 USP units per dose, from about 200 USP units per dose to about 250 USP units per dose, from about 250 USP units to about 300 USP units per dose, from about 300 USP units to about 450 USP units per dose, from about 450 USP units to about 600 USP units per dose, from about 600 USP units to about 750 USP units per dose, from about 750 USP units to about 900 USP units per dose, from about 900 USP units to about 1050 USP units per dose, from about 1050 USP units to about 1200 USP units per dose, from about 1200 USP units to about 1350 USP units per dose, or from about 1350 USP units to about 1500 USP units per dose.

Some embodiments provide a method of administering hyaluronidase to a subject, once about every 5, 10, 12, 15, 20 or 24 hrs. In some embodiments, the hyaluronidase is administered to a subject at least 1, 2, 3, 4, 5, 6, 8, or 12 times daily. In some embodiments, the large diameter synthetic membrane vesicles having encapsulated therein a therapeutic agent are administered to a subject between each administration of hyaluronidase. For example, administered to a subject at least 1, 2, 3, 4, 5, 6, 8, or 12 times daily where each administration follows an administration of hyaluronidase. In some embodiments, the large diameter synthetic membrane vesicles having encapsulated therein a therapeutic agent are administered to a subject after at least 2, 3, 4, 5, or 6 administrations of hyaluronidase. For example, the large diameter synthetic membrane vesicles having encapsulated therein a therapeutic agent can be administered 1, 2, 3, or 4 times daily and the hyaluronidase can be administered to a subject at least 6, 8, or 12 times daily. In some embodiments, 2, 3, 4, 5, 6 or more doses of large diameter synthetic membrane vesicles having encapsulated therein a therapeutic agent are administered to a subject following an administration of hyaluronidase, where each of the doses of large diameter synthetic membrane vesicles is separated by a period of time. In some embodiments the period of time is at least, or is at least about, 1 minute, 5 minutes, 10 minutes, 15 minutes, 30 or minutes. In other embodiments, the period of time is at least, or is at least about, 1, 2, 3, 4, 5, 6, 10, 12, or 15 hours. In some embodiments, the period of time is not more than about 20 hours. In some embodiments, the period is a range defined by any of the preceding values, e.g., 15 minutes to 2 hours.

### Synthetic Methods

Some embodiments for formulating the large diameter synthetic membrane vesicles, in particular multivesicular liposomes, provide making a “water-in-oil” emulsion by (1) dissolving amphipathic lipids in one or more volatile organic solvents for the lipid component, (2) adding to the lipid component an immiscible first aqueous component and a substance to be encapsulated, and (3) emulsifying the mixture mechanically.

In the emulsion, the water droplets suspended in the organic solvent will form the internal aqueous chambers, and the monolayer of amphipathic lipids lining the aqueous chambers will become one leaflet of the bilayer membrane in the final product. The emulsion can then immersed in a second aqueous component.

Then the emulsion is agitated either mechanically, by ultrasonic energy, nozzle atomizations, and the like, or by combinations thereof, to form solvent spherules suspended in the second aqueous component.

The solvent spherules contain multiple aqueous droplets with the substance to be encapsulated dissolved in them. The organic solvent is removed from the spherules, preferably by evaporation of a volatile solvent, for instance by passing a stream of gas over the suspension. When the solvent is completely removed, the spherules convert into multivesicular liposomes. Representative gases satisfactory for use in evaporating the solvent include nitrogen, helium, argon, oxygen, hydrogen, and carbon dioxide.

Many different types of volatile hydrophobic solvents such as ethers, hydrocarbons, halogenated hydrocarbons, or Freons may be used as the lipid-phase solvent. For example, diethyl ether, isopropyl and other ethers, chloroform, tetrahydrofuran, halogenated ethers, esters and combinations thereof are satisfactory.

In order to prevent the solvent spherules from sticking to each other and to the vessel wall, it is preferred that an amphipathic lipid with a net negative charge or a net positive charge be included in the spherules, and, optionally an acid may be used. In addition, one or more nonionic osmotic agents, such as trehalose, glucose, or sucrose, may optionally be used in the suspending aqueous solution to keep the osmotic pressure within and without the membrane vesicles balanced.

The second aqueous component is an aqueous solution optionally containing low ionic strength solutes such as carbohydrates including glucose, sucrose, lactose, and amino acids such as lysine, free-base histidine and combinations thereof.

For making multivesicular liposomes, it is preferred that at least one amphipathic lipid and one neutral lipid be included in the lipid component. The amphipathic lipids can be zwitterionic, anionic, or cationic lipids. Additionally, cholesterol or plant sterols can be used in making multivesicular liposomes.

Many and varied therapeutic agents can be incorporated by encapsulation within the synthetic membrane vesicles. A non-limiting list of therapeutic agent classes include, but are not limited to, antianginas, antiarrhythmics, antiasthmatic agents, antibiotics, antidiabetics, antifungals, antihistamines, antihypertensives, antiparasitics, antineoplastics, antiviral agents, otologicals, cardiac glycosides, hormones, immunomodulators, monoclonal antibodies, neurotransmitters, sedatives, vaccines, vasopressors, anesthetics, amide anaesthetics, corticosteroids, tricyclic antidepressants, tetracyclic antidepressants, selective serotonin reuptake inhibitors, steroid receptor modulators, antipsychotic drugs, antiprotozoal drugs, opioids, antiproliferative agents, salicylanilides, antihelminthic drugs, vinca alkaloids, anti-inflammatory agents, anti-depressants, prostaglandins, phosphodiesterase IV inhibitors; retinoids, steroids, β-adrenergic receptor ligands, anti-mitotic agents, microtubule inhibitors, microtubule-stabilizing agents, serotonin norepinephrine reuptake inhibitors, noradrenaline reuptake inhibitors, non-steroidal immunophilin-dependent immunosuppressants, non-steroidal immunophilin-dependent immunosuppressant enhancers; antimalarial agents, analgesics, immunosuppressants, expectorants, sulfa drugs, cardiovascular drugs, central nervous system (CNS) depressants, H2-blockers, anti-platelet drugs, anticonvulsants, alpha blockers, beta-blockers, cholinesterase inhibitors, calcium channel blockers, H1-receptor antagonists, and proteinaceous materials. The therapeutic agents listed herein can be used in the preparation of medicaments for the treatment of a disease for which the therapeutic agent is known to those of skill in the art to be effective. Therapeutic agents, and diseases for which the therapeutic agent is effective, can be identified by reference to, for example, The Physician's Desk Reference, which is incorporated herein by reference in its entirety.

Examples of proteinaceous materials that can be incorporated into the synthetic membrane vesicles, include but are not limited to, DNA, RNA, proteins of various types, protein hormones produced by recombinant DNA technology effective in humans, hematopoietic growth factors, monokines, lymphokines, tumor necrosis factor, inhibin, tumor growth factor alpha and beta, Mullerian inhibitory substance, nerve growth factor, fibroblast growth factor, platelet-derived growth factor, pituitary and hypophyseal hormones including LH and other releasing hormones.

Examples of antiarrhythmics, include but are not limited to, quinidine, procainamide, disopyramide, ajmaline, lidocaine, tocainide, mexiletine, flecainide, propafenone, moricizine, propranolol, esmolol, timolol, metoprolol, atenolol, amiodarone, sotalol, ibutilide, dofetilide, verapamil, diltiazem, and digoxin.

Examples of antiasthmatic agents, include but are not limited to, salbutamol, levalbuterol, terbutaline, bitolterol, epinephrine, ipratropium bromide, salmeterol, formoterol, bambuterol, and albuterol.

Examples of antibiotics, include but are not limited to, amikacin, gentamicin, kanamycin, neomycin, netilmicin, streptomycin, tobramycin, paromomycin, geldanamycin, herbimycin, loracarbef, ertapenem, doripenem, imipenem, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, bacitracin, colistin, polymyxin b, ciprofloxacin, enoxacin, gatifloxacin, levofloxacin, lomefloxacin, moxifloxacin, norfloxacin, ofloxacin, trovafloxacin, grepafloxacin, sparfloxacin, mafenide, prontosil, sulfacetamide, sulfamethizole, sulfanilimide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin, rifampin, thiamphenicol, and tinidazole.

Examples of antidiabetics, include but are not limited to, tolbutamide, acetohexamide, tolazamide, chlorpropamide, glipizide, glyburide, glimepiride, gliclazide, repaglinide, nateglinide, metformin, rosiglitazone, pioglitazone, troglitazone, miglitol, acarbose, exenatide, liraglutide, taspoglatide, vildagliptin, and sitagliptin.

Examples of antifungals, include but are not limited to, natamycin, rimocidin, filipin, nystatin, amphotericin B, candicin, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, abafungin, terbinafine, amorolfine, naftifine, butenafine, anidulafungin, caspofungin, micafungin, ciclopirox, tolnaftate, undecylenic acid, 5-fluorocytosine, and griseofulvin.

Examples of antihistamines, include but are not limited to, aceprometazine, alimemazine, astemizole, azatadine, azelastine, benadryl, bepotastine, bisulepine, brompheniramine, chlorcyclizine, chloropyramine, chlorothen, chlorphenamine, cinnarizine, clemastine, clemizole, clobenzepam, clobenztropine, clocinizine, cyclizine, cyproheptadine, dacemazine, dexbrompheniramine, dexchlorpheniramine, diphenhydramine, doxylamine, drixoral, ebastine, embramine, emedastine, epinastine, etymemazine, fexofenadine, homochlorcyclizine, hydroxyzine, iproheptine, isopromethazine, ketotifen, levocabastine, mebhydrolin, mepyramine, methafurylene, methapyrilene, methdilazine, moxastine, p-methyldiphenhydramine, pemirolast, pheniramine, phenyltoloxamine, resporal, rondec, semprex-d, setastine, sominex, talastine, terfenadine, thenyldiamine, thiazinamium, and triprolidine.

Examples of antihypertensives, include but are not limited to, bumetanide, ethacrynic acid, furosemide, torsemidet, epitizide, hydrochlorothiazide, chlorothiazide, bendroflumethiazide, indapamide, chlorthalidone, metolazone, amiloride, triamterene, spironolactone, atenolol, metoprolol, nadolol, oxprenolol, pindolol, propranolol, timolol, doxazosin, phentolamine, indoramin, phenoxybenzamine, prazosin, terazosin, tolazoline, bucindolol, carvedilol, labetalol, clonidine, methyldopa, guanfacine, amlodipine, felodipine, isradipine, lercanidipine, nicardipine, nifedipine, nimodipine, nitrendipine, diltiazem, verapamil, captopril, enalapril, fosinopril, lisinopril, perindopril, quinapril, ramipril, trandolapril, benazepril, candesartan, eprosartan, irbesartan, losartan, olmesartan, telmisartan, valsartan, eplerenone, spironolactone, sodium nitroprusside, clonidine, guanabenz, methyldopa, moxonidine, guanethidine, and reserpine.

Examples of antiparasitics, include but are not limited to, mebendazole, pyrantel pamoate, thiabendazole, diethycarbazine, niclosamide, praziquantel, rifampin, amphotericin B, and melarsoprol.

Examples of antineoplastics, include but are not limited to, aclarubicin, altretamine, aminopterin, amrubicin, azacitidine, azathioprine, belotecan, busulfan, camptothecin, capecitabine, carboplatin, carmofur, carmustine, chlorambucil, cisplatin, cladribine, clofarabine, cyclophosphamide, cytarabine, daunorubicin, decitabine, doxorubicin, epirubicin, etoposide, floxuridine, fludarabine, 5-fluorouracil, fluorouracil, gemcitabine, idarubicin, ifosfamide, irinotecan, mechlorethamine, melphalan, mercaptopurine, methotrexate, mitoxantrone, nedaplatin, oxaliplatin, pemetrexed, pentostatin, pirarubicin, pixantrone, procarbazine, pyrimethamine raltitrexed, rubitecan, satraplatin, streptozocin, thioguanine, triplatin tetranitrate, teniposide, topotecan, tegafur, trimethoprim, uramustine, valrubicin, vinblastine, vincristine, vindesine, vinflunine, vinorelbine, and zorubicin.

Examples of antiviral agents, include but are not limited to, abacavir, aciclovir, acyclovir, adefovir, amantadine, amprenavir, arbidol, atazanavir, atripla, boceprevir, cidofovir, combivir, darunavir, delavirdine, didanosine, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, lamivudine, lopinavir, loviride, maraviroc, moroxydine, nelfinavir, nevirapine, nexavir, oseltamivir, penciclovir, peramivir, pleconaril, raltegravir, ribavirin, rimantadine, ritonavir, saquinavir, stavudine, tenofovir, tenofovir disoproxil, tipranavir, trifluridine, trizivir, tromantadine, valaciclovir, valganciclovir, vicriviroc, vidarabine, viramidine, zalcitabine, zanamivir, and zidovudine.

Examples of otologicals, include but are not limited to, betamethasone, chloramphenicol, chlorhexidine, clioquinol, dexamethasone, gentamicin, hydrocortisone, lidocaine, miconazole, neomycin, nitrofural, polymyxin b, prednisolone, rifamycin, and tetracycline.

Examples of cardiac glycosides, include but are not limited to, digitoxin, digoxin, and deslanoside.

Examples of hormones, include but are not limited to, adiponectin, adrenocorticotropic hormone, aldosterone, androstenedione, angiotensinogen, angiotensin, antidiuretic hormone, antimullerian hormone, atrial-natriuretic peptide, brain natriuretic peptide, 25-hydroxyvitamin D₃, calcitonin, 1,25-dihydroxyvitamin D₃, cholecystokinin, corticotropin-releasing hormone, cortisol, dehydroepiandrosterone, dihydrotestosterone, dopamine, endothelin, enkephalin, epinephrine, erythropoietin, estradiol, estriol, estrone, follicle-stimulating hormone, gastrin, ghrelin, glucagon, gonadotropin-releasing hormone, growth hormone, growth hormone-releasing hormone, histamine, human chorionic gonadotropin, human placental lactogen, inhibin, insulin, insulin-like growth factor, leptin, leukotrienes, lipotropin, luteinizing hormone, melanocyte stimulating hormone, melatonin, neuropeptide y, norepinephrine, orexin, oxytocin, pancreatic polypeptide, parathyroid hormone, progesterone, prolactin, prolactin releasing hormone, prostacyclin, prostaglandins, relaxin, renin, secretin, serotonin, somatostatin, testosterone, thrombopoietin, thromboxane, thyroid-stimulating hormone, thyrotropin-releasing hormone, thyroxine, and triiodothyronine.

Examples of immunomodulators, include but are not limited to, abatacept, abetimus, adalimumab, afelimomab, aflibercept, afutuzumab, alefacept, anakinra, aselizumab, atlizumab, atorolimumab, azathioprine, basiliximab, belatacept, belimumab,bertilimumab, cedelizumab, clenoliximab, certolizumab pegol, ciclosporin, daclizumab, deforolimus, dorlimomab aritox, dorlixizumab, efalizumab, erlizumab, elsilimomab, etanercept, everolimus, faralimomab, fontolizumab, galiximab, gantenerumab, gavilimomab golimumab, gomiliximab, gusperimus, infliximab, inolimomab, ipilimumab keliximab, lebrilizumab, leflunomide, lenalidomide, lerdelimumab, lumiliximab, maslimomab, mepolizumab, metelimumab, methotrexate, morolimumab, muromonab-cd3, mycophenolic acid, natalizumab, nerelimomab, ocrelizumab, odulimomab, omalizumab, otelixizumab, pascolizumab, pexelizumab, pimecrolimus, reslizumab, rilonacept, rovelizumab, ruplizumab, siplizumab, sirolimus, tacrolimus, talizumab, telimomab aritox, temsirolimus, teneliximab, teplizumab, teriflunomide, thalidomide, tocilizumab, toralizumab, tremelimumab, ustekinumab, vapaliximab, vepalimomab, visilizumab, zanolimumab, ziralimumab, zolimomab aritox, zotarolimus, and tetrachlorodecaoxide.

Examples of monoclonal antibodies, include but are not limited to, abagovomab, abatacept, abciximab, adalimumab, adecatumumab, aflibercept, afutuzumab, alacizumab pegol, alemtuzumab, altumomab, afelimomab, anatumomab mafenatox, anrukinzumab, apolizumab, arcitumomab, aselizumab, atlizumab, atorolimumab, bapineuzumab, basiliximab, bavituximab, bectumomab, belatacept, belimumab, bertilimumab, besilesomab, bevacizumab, biciromab brallobarbital, bivatuzumab mertansine, blinatumomab, briakinumab, canakinumab, cantuzumab mertansine, capromab pendetide, catumaxomab, cedelizumab, certolizumab pegol, cetuximab, citatuzumab bogatox, cixutumumab, clenoliximab, golimumab, ustekinumab, conatumumab, dacetuzumab, dacliximab, daclizumab, denosumab, detumomab, dorlimomab aritox, dorlixizumab, ecromeximab, eculizumab, edobacomab, edrecolomab, efalizumab, efungumab, elsilimomab, enlimomab pegol, epitumomab cituxetan, epratuzumab, erlizumab, ertumaxomab, etanercept, etaracizumab, exbivirumab, fanolesomab, faralimomab, felvizumab, fezakinumab, figitumumab, fontolizumab, foravirumab, galiximab, gantenerumab, gavilimomab, gemtuzumab ozogamicin, golimumab, gomiliximab, ibalizumab, ibritumomab tiuxetan, igovomab, imciromab, infliximab, intetumumab, inolimomab, inotuzumab ozogamicin, ibalizumab, ipilimumab, iratumumab, keliximab, labetuzumab, lemalesomab, lebrilizumab, lerdelimumab, lexatumumab, libivirumab, lintuzumab, lucatumumab, lumiliximab, mapatumumab, maslimomab, matuzumab, mepolizumab, metelimumab, milatuzumab, minretumomab, mitumomab, morolimumab, motavizumab, muromonab, stamulumab, nacolomab tafenatox, naptumomab estafenatox, natalizumab, nebacumab, necitumumab, nerelimomab, nimotuzumab, nofetumomab merpentan, ocrelizumab, odulimomab, ofatumumab, omalizumab, oportuzumab monatox, oregovomab, otelixizumab, pagibaximab, palivizumab, panitumumab, panobacumab, pascolizumab, pemtumomab, pertuzumab, pexelizumab, pintumomab, priliximab, pritumumab, rafivirumab, ramucirumab, ranibizumab, raxibacumab, regavirumab, reslizumab, rilonacept, rilotumumab, rituximab, robatumumab, rovelizumab, rozrolimupab, ruplizumab, satumomab, sevirumab, sibrotuzumab, siltuximab, siplizumab, solanezumab, sonepcizumab, sontuzumab, stamulumab, sulesomab, tacatuzumab tetraxetan, tadocizumab, talizumab, tanezumab, taplitumomab paptox, tefibazumab, telimomab aritox, tenatumomab, teneliximab, teplizumab, ticilimumab, tigatuzumab, tocilizumab, toralizumab, tositumomab, trastuzumab, tremelimumab, tucotuzumab celmoleukin, tuvirumab, urtoxazumab, ustekinumab, vapaliximab, vedolizumab, veltuzumab, vepalimomab, visilizumab, volociximab, votumumab, zalutumumab, zanolimumab, ziralimumab, and zolimomab aritox.

Examples of neurotransmitters, include but are not limited to, acetylcholine, adenosine, adenosine-5′-triphosphate, aspartate, norepinephrine, dopamine, glycine, serotonin, melatonin, histamine, glutamate, gamma aminobutyric acid, and guanosine-5′-triphosphate.

Examples of sedatives, include but are not limited to, alprazolam, amobarbital, carisoprodol, chlordiazepoxide, clomethiazole, clonazepam, diazepam, diphenhydramine, estazolam, eszopiclone, ethchlorvynol, flunitrazepam, gamma-hydroxybutyrate, glutethimide, ketamine, lorazepam, methaqualone, methyprylon, midazolam, nitrazepam, oxazepam, pentobarbital, phenobarbitoltriazolam, ramelteon, secobarbital, temazepam, thalidomide, zaleplon, zolpidem, and zopiclone.

Examples of vaccines, include but are not limited to, measles vaccine, mumps vaccine, rubella vaccine, varicella vaccine, inactivated polio vaccine, inactivated influenza vaccine, influenza a virus subtype H1N1 vaccine, diphtheria toxoid vaccine, tetanus toxoid vaccine, haemophilus influenzae type B vaccine, hepatitis B vaccine, hepatitis A vaccine, and pneumoccocal conjugate vaccine.

Examples of vasopressors, include but are not limited to, epinephrine, phenylephrine, dobutamine, isoproterenol, norepinephrine, aceprometazine, alimemazine, astemizole, azatadine, azelastine, benadryl, bepotastine, bisulepine, brompheniramine, chlorcyclizine, chloropyramine, chlorothen, chlorphenamine, cinnarizine, clemastine, clemizole, clobenzepam, clobenztropine, clocinizine, cyclizine, cyproheptadine, dacemazine, dexbrompheniramine, dexchlorpheniramine, diphenhydramine, doxylamine, drixoral, ebastine, embramine, emedastine, epinastine, etymemazine, fexofenadine, homochlorcyclizine, hydroxyzine, iproheptine, isopromethazine, ketotifen, levocabastine, mebhydrolin, mepyramine, methafurylene, methapyrilene, methdilazine, moxastine, p-methyldiphenhydramine, pemirolast, pheniramine, phenyltoloxamine, resporal, rondec, semprex-d, setastine, sominex, talastine, terfenadine, thenyldiamine, thiazinamium, and triprolidine.

Examples of anesthetics, include but are not limited to, propofol, etomidate, methohexital and sodium thiopental, midazolam, diazepam, and ketamine, benzocaine, chloroprocaine, cocaine, cyclomethycaine, dimethocaine, propoxycaine, procaine, proparacaine, tetracaine, articaine, bupivacaine, carticaine, dibucaine, etidocaine, levobupivacaine, lidocaine, mepivacaine, piperocaine, prilocaine, ropivacaine, trimecaine, saxitoxin, and tetrodotoxin.

Examples of amide anesthetics, include but are not limited to, articaine, bupivacaine, carticaine, dibucaine, etidocaine, levobupivacaine, lidocaine, mepivacaine, piperocaine, prilocaine, ropivacaine, and trimecaine.

Examples of corticosteroids, include but are not limited to, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate.

Examples of tricyclic antidepressants, include but are not limited to, amitriptyline, butriptyline, clomipramine, dosulepin, doxepin, imipramine, lofepramine, trimipramine, desipramine, nortriptyline, and protriptyline.

Examples of tetracyclic antidepressants, include but are not limited to, amoxapine, maprotiline, mianserin, mirtazapine, and setiptiline.

Examples of selective serotonin reuptake inhibitors, include but are not limited to, citalopram, dapoxetine, escitalopram, fluoxetine, fluvoxamine, paroxetine, sertraline, vilazodone, and zimelidine.

Examples of antipsychotic drugs, include but are not limited to, haloperidol, droperidol, chlorpromazine, fluphenazine, perphenazine, prochlorperazine, thioridazine, trifluoperazine, mesoridazine, periciazine, promazine, triflupromazine, levomepromazine, promethazine, pimozide, chlorprothixene, flupenthixol, thiothixene, zuclopenthixol, clozapine, olanzapine, risperidone, quetiapine, ziprasidone, amisulpride, asenapine, paliperidone, aripiprazole, and bifeprunox.

Examples of antiprotozoal drugs, include but are not limited to, eflornithine, furazolidone, melarsoprol, metronidazole, ornidazole, paromomycin sulfate, pentamidine, pyrimethamine, and tinidazole.

Examples of opioids, include but are not limited to, endorphins, enkephalins, dynorphins, endomorphins, codeine, morphine, thebaine, oripavine, diacetylmorphine, dihydrocodeine, hydrocodone, hydromorphone, nicomorphine, oxycodone, oxymorphone, fentanyl, alphamethylfentanyl, alfentanil, sufentanil, remifentanil, carfentanyl, ohmefentanyl, pethidine, ketobemidone, allylprodine, prodine, propoxyphene, dextropropoxyphene, dextromoramide, bezitramide, piritramide, methadone, dipipanone, levomethadyl acetate, loperamide, diphenoxylate, dezocine, pentazocine, phenazocine, buprenorphine, dihydroetorphine, etorphine, butorphanol, nalbuphine, levorphanol, levomethorphan, lefetamine, meptazinol, tilidine, tramadol, tapentadol, nalmefene, naloxone, and naltrexone.

Examples of antiproliferative agents, include but are not limited to, aclarubicin, altretamine, aminopterin, amrubicin, azacitidine, azathioprine, belotecan, busulfan, camptothecin, capecitabine, carboplatin, carmofur, carmustine, chlorambucil, cisplatin, cladribine, clofarabine, cyclophosphamide, cytarabine, daunorubicin, decitabine, docetaxel, doxorubicin, epirubicin, etoposide, floxuridine, fludarabine, 5-fluorouracil, fluorouracil, gemcitabine, idarubicin, ifosfamide, irinotecan, mechlorethamine, melphalan, mercaptopurine, methotrexate, mitoxantrone, nedaplatin, oxaliplatin, paclitaxel, pemetrexed, pentostatin, pirarubicin, pixantrone, procarbazine, pyrimethamine raltitrexed, rubitecan, satraplatin, sirolimus, streptozocin, thioguanine, triplatin tetranitrate, teniposide, topotecan, tegafur, trimethoprim, uramustine, valrubicin, vinblastine, vincristine, vindesine, vinflunine, vinorelbine, and zorubicin.

Examples of salicylanilides, include but are not limited to, niclosamide, oxyclozanide, and rafoxanide.

Examples of antihelminthic drugs, include but are not limited to, abamectin, albendazole, diethylcarbamazine, mebendazole, niclosamide, ivermectin, suramin, thiabendazole, pyrantel pamoate, levamisole, praziquantel, triclabendazole, flubendazole, fenbendazole, emodepside, and monepantel.

Examples of vinca alkaloids, include but are not limited to, vinblastine, vincristine, vindesine and vinorelbine.

Examples of anti-inflammatory agents, include but are not limited to, phenylbutazone, mofebutazone, oxyphenbutazone, clofezone, kebuzone, indometacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, piroxicam, tenoxicam, droxicam, lornoxicam, meloxicam, ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, mefenamic acid, tolfenamic acid, flufenamic acid, meclofenamic acid, celecoxib, rofecoxib, valdecoxib, parecoxib, etoricoxib, lumiracoxib, nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, and chondroitin sulfate.

Examples of cancers that can be treated with an anticancer agent include, but are not limited to, head and neck cancer, breast cancer, colorectal cancer, gastric cancer, hepatic cancer, bladder cancer, cervical cancer, endometrial cancer, lung cancer (non-small cell), ovarian cancer, pancreatic cancer, prostate cancer; choriocarcinoma (lung cancer); hairy cell leukemia, chronic lymphotic leukemia, acute lymphocytic leukemia (breast & bladder), acute myelogenous leukemia, Hodgkin's lymphoma, non-Hodgkin's lymphoma (osteogenic sarcoma, adult soft tissue sarcoma), meningeal leukemia, multiple myeloma, chronic myelogenous leukemia, erythroleukemia, and T-cell lymphoma.

Examples of inflammatory and autimmune diseases that can be treated with an inflammatory agent include, but are not limited to, B cell disorders, T cell disorders, Rheumatoid arthritis (RA), Systemic Lupus Erythematosus (SLE), Sjogren's syndrome, Immune thrombocytopenic purpura (ITP), Multiple sclerosis (MS), Myasthenia Gravis (MG), Graves disease, Psoriasis, Hashimoto's disease, Immune Thrombocytopenic purpura, Scleroderma, and Inflamatory Bowel Disease (e.g. Crohn's disease and ulcerative colitis).

### Definitions

As used herein, the term “large diameter synthetic membrane vesicles” means man-made, microscopic lipid-vesicles consisting of lipid bilayer membranes. The large diameter synthetic membrane vesicles can be multivesicular liposomes, unilamellar liposomes or multilamellar liposomes. Preferably, the large diameter synthetic membrane vesicles are multivesicular liposomes, unilamellar liposomes or multilamellar liposomes having an average diameter of at least 500 nm. More preferably, the large diameter synthetic membrane vesicles are multivesicular liposomes having an average diameter of at least 1000 nm. For example, the multivesicular liposomes can have an average diameter of at least, or at least about, 1 μm, 5 μm, 10 μm, 15 μm, 20 μm, 50 μm, or 100 μm, or a diameter within a range defined by any of two of the preceding values.

As used herein, the term “multivesicular liposomes” as used throughout the specification and claims means manufactured, microscopic lipid-vesicles enclosing multiple non-concentric aqueous chambers formed by internal membranes distributed as a network throughout the multivesicular liposomes. The multivesicular liposomes are typically approximately 20 μm in diameter but are not limited to this size. In contrast, unilamellar vesicles have a single aqueous chamber; and multilamellar liposomes have multiple “onion-skin” type of concentric membranes, in between which are concentric aqueous compartments.

As used herein, the term “solvent spherule” as used throughout the specification and claims means a microscopic spheroid droplet of organic solvent, within which are suspended multiple smaller droplets of aqueous solution.

As used herein, the term “neutral lipid” means oils or fats that have no membrane-forming capability by themselves and lack a hydrophilic “head” group. Examples of neutral lipids, include but are not limited to, diglycerides, such as diolein, dipalmitolein, and mixed caprylin-caprin diglycerides; triglycerides, such as triolein, tripalmitolein, trilinolein, tricaprylin, and trilaurin; vegetable oils, such as soybean oil; animal fats, such as lard and beef fat; squalene; tocopherol; and combinations thereof.

As used herein, the term “amphipathic lipids” means those molecules that have a hydrophilic “head” group and hydrophobic “tail” group and have membrane-forming capability. Examples of amphipathic lipids, include but are not limited to, 1,2-dioleoyl-sn-glycero-3-phosphocholine, 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1,2-diarachidoyl-sn-glycero-phosphocholine, 1,2-dibehenoyl-sn-glycero-3-phosphocholine, 1,2-dipalmitoleoyl-sn-glycero-3-phosphocholine, 1,2-dieicosenoyl-sn-glycero-3-phosphocholine, 1,2-dierucoyl-sn-glycero-3-phosphocholine, 1,2-dipalmitoyl-sn-glycero-3-phosphoglycerol and 1,2-dioleoyl-sn-glycero-3-phosphoglycerol.

As used herein, the term “zwitterionic lipid” means an amphipathic lipid with a net charge of zero at pH 7.4. Examples of zwitterionic amphipathic lipids, include but are not limited to, phosphatidylcholines, phosphatidylethanolamines, sphingomyelins and the like.

As used herein, the term “anionic lipid” means an amphipathic lipid with a net negative charge at pH 7.4. Examples of anionic amphipathic lipids, include but are not limited to, phosphatidylglycerols, phosphatidylserines, phosphatidylinositols, phosphatidic acids, and the like.

As used herein, the term “cationic lipid” means an amphipathic lipid with a net positive charge at pH 7.4. Examples of cationic amphipathic lipids, include but are not limited to, diacyl trimethylammoniumpropane, ethyl phosphatidylcholine and the like.

As used herein, the term “therapeutic agent” includes any natural or synthetic substance intended to provide benefit to the subject administered the substance, including the treatment or prevention of a disease.

As used herein, the term “solvent spherule” means a microscopic spheroid droplet of organic solvent, within which is multiple smaller droplets of aqueous solution. The solvent spherules are suspended and totally immersed in a second aqueous solution.

As used herein, the term “neutral lipid” means oil or fats that have no membrane-forming capability by themselves and lack a hydrophilic “head” group.

As used herein, the term “amphipathic lipids” means those molecules that have a hydrophilic “head” group and hydrophobic “tail” group and have membrane-forming capability.

### Additional Embodiments

Some embodiments provide a pharmaceutical composition, comprising hyaluronidase; and large diameter synthetic membrane vesicles, the large diameter synthetic membrane vesicles having encapsulated therein a therapeutic agent. In some embodiments, the hyaluronidase is encapsulated in large diameter synthetic membrane vesicles. In other embodiments the large diameter synthetic membrane vesicles encapsulate the hyaluronidase.

In some embodiments, the large diameter synthetic membrane vesicles have an average diameter selected from the group consisting of at least about 0.5 μm, 1 μm, 5 μm, 10 μm, 15 μm, 20 μm, 50 μm, and 100 μm. In some embodiments, the large diameter synthetic membrane vesicles have an average diameter of at least about 10 μm. In other embodiments, the large diameter synthetic membrane vesicles have an average diameter between about 1 μm and about 50 μm.

Some embodiments provide a pharmaceutical composition of any of the disclosed embodiments where the therapeutic agent comprises one or more drugs. Other embodiments provide a pharmaceutical composition of any of the embodiments disclosed herein further comprising a pharmaceutically acceptable carrier. Some embodiments provide a pharmaceutical composition of any of the embodiments disclosed herein where the hyaluronidase is rHuPH20.

Some embodiments provide a method of administering a therapeutic agent to a subject, comprising administering the pharmaceutical composition of any of the disclosed embodiments.

Some embodiments provide a method of administering a therapeutic agent to a subject, comprising mixing a hyaluronidase and large diameter synthetic membrane vesicles, the large diameter synthetic membrane vesicles having encapsulated therein a therapeutic agent; and administering the mixture of the hyaluronidase and large diameter synthetic membrane vesicles to the subject. In some embodiments, the administration is subcutaneous.

Some embodiments provide a method of administering a therapeutic agent to a subject, the method comprising administering a hyaluronidase to a subject; and, administering large diameter synthetic membrane vesicles to a subject, the large diameter synthetic membrane vesicles having encapsulated therein a therapeutic agent. In some embodiments, administration of the large diameter synthetic membrane vesicles follows administration of the hyaluronidase after a period of time. In other embodiments, the hyaluronidase and the large diameter synthetic membrane vesicles are administered concurrently. In other embodiments, the method further comprises administering additional large diameter synthetic membrane vesicle encapsulating a therapeutic agent to the subject after a period of time following the concurrent administration of the hyaluronidase the large diameter synthetic membrane vesicles.

Some embodiments provide a method where the period of time is at least, or is at least about, 1 minute, 5 minutes, 10 minutes, 15 minutes, 30 or minutes. In other embodiments, the period of time is at least, or is at least about, 1, 2, 3, 4, 5, 6, 10, 12, or 15 hours. In some embodiments, the period of time is not more than about 20 hours. In some embodiments, the period is a range defined by any of the preceding values, e.g., 15 minutes to 2 hours.

Some embodiments provide a method where the administration of hyaluronidase is subcutaneous. In some embodiments the administration of large diameter synthetic membrane vesicles is subcutaneous. In some embodiments, the hyaluronidase is administered by bolus injection. In some embodiments, the large diameter synthetic membrane vesicles are administered by infusion or injection. In some embodiments, the hyaluronidase is administered by a single bolus injection and the large diameter synthetic membrane vesicles are administered by infusion. Some embodiments provide a method where the hyaluronidase is administered by infusion and the large diameter synthetic membrane vesicles are administered by infusion or a single bolus injection.

Some embodiments provide a method where the site of administration of hyaluronidase and the site of administration of large diameter synthetic membrane vesicles are the same.

Some embodiments provide a method where the large diameter synthetic membrane vesicles have an average diameter selected from the group consisting of at least about 0.5 μm, 1 μm, 5 μm, 10 μm, 15 μm, 20 μm, 50 μm, and 100 μm.

Some embodiments provide a kit comprising a syringe comprising the pharmaceutical composition of any of the disclosed embodiments. Other embodiments provide a kit comprising a sterile, single use container comprising the composition of any of the disclosed embodiments. Additional embodiments provide a kit disclosed herein, and a syringe adapted to receive the composition from the container.

Some embodiments provide a kit comprising a syringe containing a hyaluronidase, large diameter synthetic membrane vesicles and a therapeutic agent. Other embodiments provide a kit where the syringe has a single chamber; other embodiments provide a kit where the syringe has two chambers. Further embodiments provide a kit where one chamber comprises the large diameter synthetic membrane vesicles and the therapeutic agent and the other chamber comprises hyaluronidase.

Some embodiments provide a kit comprising a sterile container comprising hyaluronidase and large diameter synthetic membrane vesicles, the large diameter synthetic membrane vesicles having encapsulated therein a therapeutic agent. Some embodiments provide a kit where the container is a single use container. Some embodiments provide a kit where the container is a multi-use container further comprising a preservative.

Some embodiments provide a kit comprising a sterile single use container comprising large diameter synthetic membrane vesicles, the large diameter synthetic membrane vesicles having encapsulated therein a therapeutic agent, and, a sterile single use container comprising hyaluronidase.

Some embodiments provide a kit comprising a multi-use container comprising preservative and large diameter synthetic membrane vesicles, the large diameter synthetic membrane vesicles having encapsulated therein a therapeutic agent; and, a sterile single use container comprising hyaluronidase.

Some embodiments provide a kit comprising a first multi-use container comprising preservative and large diameter synthetic membrane vesicles, the large diameter synthetic membrane vesicles having encapsulated therein a therapeutic agent, and, a second multi-use container comprising preservative and hyaluronidase. Further embodiments provide a kit where the large diameter synthetic membrane vesicles have an average diameter selected from the group consisting of at least about 0.5 μm, 1 μm, 5 μm, 10 μm, 20 μm, 50 μm, and 100 μm. Additional embodiments provide a kit where the hyaluronidase is rHuPH2O.

### NONLIMITING DISCLOSURE AND INCORPORATION BY REFERENCE

While certain therapeutic agents, compositions and methods of the present invention have been described with specificity in accordance with certain embodiments, the following examples serve only to illustrate the compositions and methods of the invention and are not intended to limit the same. Each of the references, patents, publications and the like recited herein is incorporated herein by reference for the disclosure for which it is referenced, and in its entirety.

### Examples

### Example 1

Multivesicular liposome test articles containing recombinant human hyaluronidase were generated according to the composition shown in Table I by the method of Kim, et al. (Biochim, Biophys. Acta, 728:339-348, 1983) with the exception that the emulsions were formed by mechanical mixing as opposed to mechanical shaking using volumes five-fold greater than Kim et al. In addition, chloroform was used as the solvent for the lipid phase in the work described herein. Physiologically isotonic histidine buffered sucrose was used to create the water-in-oil-in-water emulsion and conduct removal of the organic solvent. Trypan blue was added to the aqueous contents as a potential visual indicator of subcutaneous particle dispersion.

TABLE I
Lipid Composition and Aqueous Composition to make First Emulsion.

| | Initial | | Aqueous | Initial | |
|---|---|---|---|---|---|
| Lipid Components | Concentration | | Contents | Concentration | |
| Dielaidoylglycerophosphocholine | 26.4 | mM | Sterile water | >99% | |
| (DEPC) | | | for injection | | |
| Dipalmitoylphosphatidylglycerol | 11.2 | mM | Glutamate | 30 | mM |
| (DPPG) | | | | | |
| Tricaprylin | 45 | mM | Arginine | 5 | mM |

| | | | | |
|---|---|---|---|---|
| Cholesterol | 40 | mM | Sucrose | 8% |

| | | |
|---|---|---|
| Trypan Blue | 0.04% | w/v |
| Sodium Chloride | 0.018% | w/v |
| Dibasic | 0.006% | w/v |
| potassium | | |
| phosphate | | |

After isolation by centrifuge, the multivesicular liposomes were resuspended with a volume of recombinant human hyaluronidase in saline (150 Units/mL) equivalent to the mass of multivesicular liposomes collected, as shown in Table II. As a comparator, multivesicular liposomes were also generated using only saline with no recombinant human hyaluronidase. The mass of the multivesicular liposomes was determined and the multivesicular liposomes were resuspended with a mass-equivalent volume of hyaluronidase in saline at twice the desired potency. The amount of recombinant human hyaluronidase used in the resuspension provided either 15 units per 1 mL or 30 units per 1 mL. The final hyaluronidase potency was therefore achieved via dilution.

TABLE II
Components For Resuspension of the Multivesicular liposomes

| Storage Solution | Concentration |
|---|---|
| Sterile saline for irrigation | 0.9% w/v |
| Recombinant Human Hyaluronidase | 15 or 30 Units/mL |

In addition, the multivesicular liposomes flowed freely and did not appear to be aggregated upon visual inspection. There were no differences observed between the batches, regardless of the presence of recombinant human hyaluronidase.

The batches were stored overnight at 5±3° C. and the packed particle volume (PPV), a volumetric measurement of the particles in solution, was measured for both batches. Each batch manifested a 44% PPV suggesting that each batch contained an equivalent amount of particles.

In addition, the particle size distribution was measured the following day on both batches. There was no significant difference between the particle size distributions regardless of the presence of hyaluronidase (Table III).

TABLE III
Particle size distribution after overnight storage
at 5 ± 3° C. for batches in the absence
of hyaluronidase and in the presence of hyaluronidase.

| Hyaluronidase | D₁₀ (μm) | D₅₀ (μm) | D₉₀ (μm) | Mean (μm) |
|---|---|---|---|---|
| NO | 8.9 | 15.5 | 26.2 | 16.8 |
| YES | 8.6 | 14.9 | 25.2 | 16.1 |

There were no significant differences observed in particle morphology, packed particle volume, particle size distribution or the visual inspection of the batches. Following overnight storage at 5±3° C., no significant differences were observed, suggesting that their stability characteristics are similar over the time period studied. Together, the data suggest that multivesicular liposomes are compatible with hyaluronidase in the storage solution. Moreover, the data suggests that the presence of hyaluronidase does not induce untoward stability of the multivesicular liposomes formulation.

### Example 2

Multivesicular liposome test articles containing recombinant human hyaluronidase were generated according to the composition shown in Table I by the method of Kim, et al. (Biochim, Biophys. Acta, 728:339-348, 1983) with the exception that the emulsions were formed by mechanical mixing as opposed to mechanical shaking using volumes five-fold greater than Kim et al. In addition, chloroform was used as the solvent for the lipid phase in the work described herein. Physiologically isotonic histidine buffered sucrose was used to create the water-in-oil-in-water emulsion and conduct removal of the organic solvent. Trypan blue was added to the aqueous contents as a potential visual indicator of subcutaneous particle dispersion.

The effect of recombinant human hyaluronidase on the force of injection of a pharmaceutical composition of multivesicular liposomes was evaluated. The multivesicular liposomes were injected into a domestic pig (Sus scrofa domestica) in combination with or without recombinant human hyaluronidase. The study was designed as described below.

### Materials and Methods

One male Yorkshire pig (Sus scrofa domestica) weighing 30 to 50 kg was used. The pig age was commensurate with weight and the acclimation was 5 days. A saline control injection manufactured by B. Braun Medical, Inc. (Lot No. J8S205) was administered subcutaneously. The recombinant human hyaluronidase potency was 0 Units/mL with dose levels of 0 Units for a 1 mL dose.

The recombinant human Hyaluronidase was (HYLENEX™ Baxter Healthcare Corp. One Baxter Parkway Deerfield, Ill. 60015-4625) manufactured by Baxter Healthcasre, Corp. (Lot No. 906429). The potency was 150 Units/mL with dose levels of 150 or 300 Units (approximately 3.8 or 7.5 Units/kg).

### Experimental Design

At no point did the rate of injection exceed that for normal human intravenous infusion, or approximately 5 mL/min. The Force of Injection for the various test articles was evaluated as described in the following scale: 1=Easy, force required for delivery was equivalent to a 1.0 mL saline injection; 2=Moderate, force required was slightly more than that required for a 1.0 mL saline injection; 3=Difficult, force required was significantly more than that required for a 1.0 mL saline injection; and 4=Very Difficult, extreme force must be used for delivery.

A Force of Injection rating of 1 (Easy) was awarded when the evaluator applied a force that was comparable to the average force needed to perform routine subcutaneous injections with saline, whereas a Force of Injection rating of 4 (Very difficult) was awarded when it was not practical to inject additional solution into the animal.

The ability to visualize the dye under the skin will also be recorded as either “Yes” or “No,” with comments included as necessary.

### Injection Procedures

The injection sites were shaved, and the test articles were administered by subcutaneous injection into the back with a 10 cc disposable syringe equipped with a 25 G, 1.5″ hypodermic needle. At no point did the rate of injection exceed that for normal human intravenous infusion, or approximately 5 mL/min.

A saline control injection of 1.0 mL, manufactured by B. Braun Medical, Inc. (Lot No. J8S205), was administered subcutaneously.

Multivesicular liposomes placebo A containing recombinant human hyaluronidase in the suspension solution was resuspended by gently rolling, rotating, and inverting product vial, at least 20 times for each motion. It was then administered subcutaneously. The subject was injected with 10 mL of the multivesicular liposomes placebo containing a total of 150 units (15 units/mL) recombinant human hyaluronidase. In 2.5 mL increments, the actual volume injected, elapsed time, and the Force of Injection relative to the saline control was recorded. A rating of 1 indicates that a force equivalent to that required for injection of 1 mL saline was employed. If the entire 10 mL was delivered with a Force of Injection rating of 2 or less and the injection site can be adequately assessed, additional injections were not necessary.

Multivesicular liposomes placebo B containing recombinant human hyaluronidase in the suspension solution was resuspended by gently rolling, rotating, and inverting product vial, at least 20 times for each motion. It was then administered subcutaneously. The subject was injected with 10 mL of multivesicular liposomes placebo containing a total of 300 units (30 units/mL) recombinant human hyaluronidase. In 2.5 mL increments, the actual volume injected, elapsed time, and the Force of Injection relative to the saline control were recorded. A rating of 1 indicates that a force equivalent to that required for injection of 1 mL saline was employed. If the entire 10 mL was delivered with an injection difficulty level of 2 or less and the injection site could be adequately assessed as required, additional injections were not necessary.

Multivesicular liposomes placebo C did not contain recombinant human hyaluronidase and was resuspended by gently rolling, rotating, and inverting product vial, at least 20 times for each motion. It was then administered subcutaneously. The subject was subcutaneously injected with 10 mL multivesicular liposomes placebo without recombinant human hyaluronidase. In 2.5 mL increments, the actual volume injected, elapsed time, and the Force of Injection relative to the saline control may be recorded. A rating of 1 indicates that a force equivalent to that required for injection of 1 mL saline is employed. If the entire 10 mL was delivered with an injection difficulty level of 2 or less and the injection site could be adequately assessed as required, additional injections were not necessary.

Results of recombinant human hyaluronidase (15 Units/mL; 150 Units obtained from Baxter Healthcare Corp.) on the force of injection of a pharmaceutical composition of multivesicular liposomes are disclosed below. The force of injection was assessed over the duration of the full 10 mL of injection with reporting points after delivery of 0 to 2.5 mL, 2.5 to 5 mL, 5 to 7.5 mL and 7.5 to 10 mL. The force of injection required for each test article was assessed relative to the force required for the subcutaneous injection of 1 mL of saline. See Table IV.

TABLE IV
Force of Injection results for test article Placebo A.

| | | | | Force of Injection | | | |
|---|---|---|---|---|---|---|---|
| | | | | | (2) | (3) | |
| | | | | (1) | Moderate: | Difficult: | (4) |
| | | | | Easy: | Slightly more | Considerably | Very Difficult: |
| | | Actual Volume | Elapsed Time | Similar to | difficult than | more difficult | Extreme force |
| | | Delivered (mL) | (min) | Saline | saline | than saline | must be used |
| 0-2.5 | mL | 2.5 | ca. 30 s | | X | | |
| 2.5-5 | mL | 5 | ca. 30 s | X | | | |
| 5-7.5 | mL | 7.5 | ca. 30 s | X | | | |
| 7.5-10 | mL | 10 | ca. 30 s | X | | | |

| | | | |
|---|---|---|---|
| Total Volume | 10 | Total Elapsed | 2 |
| Delivered (mL): | | Time (min): | |

Test article contains 150 Units recombinant human Hyaluronidase in a total volume of 10 mL (15 Units/mL).

Results of recombinant human hyaluronidase (30 Units/mL) on the force of injection of a pharmaceutical composition of multivesicular liposomes is disclosed below. The force of injection was assessed over the duration of the full 10 mL injection with reporting points after the delivery of 0 to 2.5 mL, 2.5 to 5 mL, 5 to 7.5 mL and 7.5 to 10 mL. The force of injection required for each test article was assessed relative to the force required for the subcutaneous injection of 1 mL of saline. See Table IV.

TABLE V
Force of Injection results for test article Placebo B.

| | | | | Force of Injection | | | |
|---|---|---|---|---|---|---|---|
| | | | | | (2) | (3) | |
| | | | | (1) | Moderate: | Difficult: | (4) |
| | | | | Easy: | Slightly more | Considerably | Very Difficult: |
| | | Actual Volume | Elapsed Time | Similar to | difficult than | more difficult | Extreme force |
| | | Delivered (mL) | (min) | Saline | saline | than saline | must be used |
| 0-2.5 | mL | 2.5 | ca. 30 s | | X | | |
| 2.5-5 | mL | 5 | ca. 30 s | X | | | |
| 5-7.5 | mL | 7.5 | ca. 30 s | X | | | |
| 7.5-10 | mL | 10 | ca. 30 s | X | | | |

| | | | |
|---|---|---|---|
| Total Volume | 10 | Total Elapsed | 2 |
| Deliverred (mL): | | Time (min): | |

Test article contains 300 Units recombinant human Hyaluronidase in a total volume of 10 mL (30 Units/mL).

Results on the force of injection of a pharmaceutical composition of multivesicular liposomes with no recombinant human hyaluronidase is disclosed below. The force of injection was assessed over the duration of the full 10 mL injection with reporting points after the delivery of 0 to 2.5 mL, 2.5 to 5 mL, 5 to 7.5 mL and 7.5 to 10 mL. The results show that the 0 to 2.5 mL injection and the 2.5 to 5 mL injection were slightly more difficult than saline. The results show that the 5 to 7.5 mL and 7.5 to 10 mL were considerably more difficult than saline. See Table VI.

TABLE VI
Force of Injection results for test article Placebo C.

| | | | | Force of Injection | | | |
|---|---|---|---|---|---|---|---|
| | | | | | (2) | (3) | |
| | | | | (1) | Moderate: | Difficult: | (4) |
| | | | | Easy: | Slightly more | Considerably | Very Difficult: |
| | | Actual Volume | Elapsed Time | Similar to | difficult than | more difficult | Extreme force |
| | | Delivered (mL) | (min) | Saline | saline | than saline | must be used |
| 0-2.5 | mL | 2.5 | ca. 30 s | | X | | |
| 2.5-5 | mL | 5 | ca. 30 s | | X | | |
| 5-7.5 | mL | 7.5 | ca. 30 s | | | X | |
| 7.5-10 | mL | 10 | ca. 30 s | | | X | |

| | | | |
|---|---|---|---|
| Total Volume | 10 | Total Elapsed | 2 |
| Deliverred (mL: | | Time (min): | |

Test article is 10 mL of placebo only with no recombinant human Hyaluronidase present.

The results in Tables IV, V, and VI demonstrate that recombinant human hyaluronidase has an effect on the force of injection for a pharmaceutical composition of multivesicular liposomes.

### Example 3

The subject is injected with 150 units of recombinant human hyaluronidase followed by an injection of multivesicular liposomes. Here, 1 mL of recombinant human hyaluronidase (150 units/mL) is subcutaneously injected into the planned site. After waiting 10 minutes for recombinant human hyaluronidase to act, 10 mL multivesicular liposomes placebo (without recombinant human hyaluronidase) is injected into the same planned injection site. In 2.5 mL increments, the actual volume injected, elapsed time, and the Force of Injection relative to the saline control is recorded. A rating of 1 indicates that a force equivalent to that required for injection of 1 mL saline is employed. If the entire 10 mL is delivered with an injection difficulty level of 2 or less and the injection site can be adequately assessed, additional injections will not be necessary.

The subject is injected with 300 units of recombinant human hyaluronidase followed by an injection of multivesicular liposomes. Here, 300 units of recombinant human hyaluronidase (2 mL at 150 units/mL) is subcutaneously injected into the planned site. After waiting 10 minutes for recombinant human hyaluronidase to act, 10 mL multivesicular liposomes placebo (without recombinant human hyaluronidase) is injected into the same planned injection site. In 2.5 mL increments, the actual volume injected, elapsed time, and the Force of Injection relative to the saline control is recorded. A rating of 1 indicates that a force equivalent to that required for injection of 1 mL saline is employed. If the entire 10 mL was delivered with an injection difficulty level of 2 or less and the injection site can be adequately assessed as required, additional injections will not be necessary.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A pharmaceutical composition for enhancing the administration of a therapeutic agent in large diameter synthetic membrane vesicles, comprising
hyaluronidase; and
large diameter synthetic membrane vesicles, said large diameter synthetic membrane vesicles having encapsulated therein a therapeutic agent.

2. The pharmaceutical composition of claim 1, wherein said therapeutic agent is a therapeutic peptide or a proteinaceous material.

3. The pharmaceutical composition of claim 1, wherein said hyaluronidase is encapsulated in large diameter synthetic membrane vesicles.

4. The pharmaceutical composition of claim 1, wherein said large diameter synthetic membrane vesicles have an average diameter of at least 500 nm.

5. The pharmaceutical composition of claim 1, wherein said large diameter synthetic membrane vesicles have an average diameter between 1 μm and 50 μm.

6. The pharmaceutical composition of claim 1, wherein said hyaluronidase is rHuPH20.

7. A method of administering a therapeutic agent to a subject in need thereof, comprising administering the pharmaceutical composition of claim 1 into said subject.

8. The method of claim 7, wherein said composition is administered by injection.

9. The method of claim 7, wherein said administration is selected from the group consisting of subcutaneous injection, intramuscular injection and intradermal injection.

10. A method of enhancing the administration of a therapeutic agent in large diameter synthetic membrane vesicles to a subject in need thereof, comprising
administering hyaluronidase to a subject; and,
administering large diameter synthetic membrane vesicles having encapsulated therein a therapeutic agent to said subject,
wherein ease of administration of said large diameter synthetic membrane vesicles is improved, or the volume of said large diameter synthetic membrane vesicles that can be administered is increased, in comparison to administration of said large diameter synthetic membrane vesicles in the absence of said hyaluronidase.

11. The method of claim 10, wherein said hyaluronidase and said large diameter synthetic membrane vesicles are administered concurrently.

12. The method of claim 11, wherein said hyaluronidase and said large diameter synthetic membrane vesicles are administered as a mixture.

13. The method of claim 11, further comprising administering additional large diameter synthetic membrane vesicle encapsulating a therapeutic agent to said subject after a period of time following said concurrent administration of said hyaluronidase said large diameter synthetic membrane vesicles.

14. The method of claim 10, wherein administration of said large diameter synthetic membrane vesicles follows administration of said hyaluronidase after a period of time.

15. The method of claim 14, wherein said period of time is at least about 1 minute.

16. The method of claim 10, wherein said administration of said hyaluronidase and said large diameter synthetic membrane vesicles is selected from the group consisting of subcutaneous, intramuscular and intradermal.

17. The method of claim 10, wherein said hyaluronidase is administered by bolus injection or infusion and said large diameter synthetic membrane vesicles are administered by bolus injection or infusion.

18. The method of claim 10, wherein the site of administration of said hyaluronidase and the site of administration of said large diameter synthetic membrane vesicles are the same.

19. The method of claim 10, wherein the site of administration of said hyaluronidase and the site of administration of said large diameter synthetic membrane vesicles are less than 5 cm from each other.

20. The method of claim 10, wherein said hyaluronidase is encapsulated in large diameter synthetic membrane vesicles.

21. The method of claim 10, wherein said large diameter synthetic membrane vesicles have an average diameter of at least 500 nm.

22. The method of claim 10, wherein said hyaluronidase is rHuPH20.

23. A kit for enhancing the administration of a therapeutic agent in large diameter synthetic membrane vesicles comprising a first sterile container comprising large diameter synthetic membrane vesicles, said large diameter synthetic membrane vesicles having encapsulated therein a therapeutic agent, and, a second sterile container comprising hyaluronidase.

24. The kit of claim 23, wherein said first and second containers are chambers of a two chamber syringe.

25. The kit of claim 23, further comprising a third container for mixing said hyaluronidase and said large diameter synthetic membrane vesicles.

26. The kit of claim 23, wherein said hyaluronidase is rHuPH20.

27. The kit of claim 23, wherein said large diameter synthetic membrane vesicles have an average diameter of at least 500 nm.

28. A method of providing a therapeutic agent in large diameter synthetic membrane vesicles to an individual comprising:
providing said individual large diameter synthetic membrane vesicles, said large diameter synthetic membrane vesicles having encapsulated therein a therapeutic agent,
and providing said individual instructions to administer hyaluronidase prior to or concurrent with said large diameter synthetic membrane vesicles.

29. The method of claim 28, wherein said large diameter synthetic membrane vesicles have an average diameter of at least 500 nm.

30. The method of claim 28, wherein said hyaluronidase is rHuPH20.
